(19) United States
(12) Patent Application Publication
(10) Pub. No.: US 2021/0330487 A1
(43) Pub. Date: Oct. 28, 2021

(54) ORAL DEVICE

(71) Applicant: Nazar Kalivoshko, Hinckley, OH (US)

(72) Inventor: Nazar Kalivoshko, Hinckley, OH (US)

(21) Appl. No.: 17/239,881

(22) Filed: Apr. 26, 2021

Related U.S. Application Data
(60) Provisional application No. 63/015,839, filed on Apr. 27, 2020.

(51) Int. Cl. A61F 5/56; A61M 21/00

(52) U.S. Cl. CPC A61F 5/566; A61M 21/00; A61M 2021/0022; A61M 2230/435; A61M 2021/0083; A61M 2205/3303; A61M 2205/3553; A61M 2021/0055

(57) ABSTRACT

An oral device includes a mouthpiece and a stimulant package including a motor that causes the mouthpiece to vibrate. The stimulant package includes a sensor, a battery, and a controller including a transceiver communicating with a remote device by a wired or wireless connection. The sensor and/or the remote device detect specified conditions for activating the stimulant package. Programming and/or control are performed at a user interface of the stimulant package and/or the remote device. Inhibiting snoring and/or sleep apnea includes placing the device in a user's mouth prior to sleeping and activating the motor to cause the mouthpiece to vibrate when a specified condition is met. Waking a user includes setting a parameter including a time of day; placing the device in a user's mouth prior to sleeping; and activating the motor to cause the mouthpiece to vibrate at the time of day.

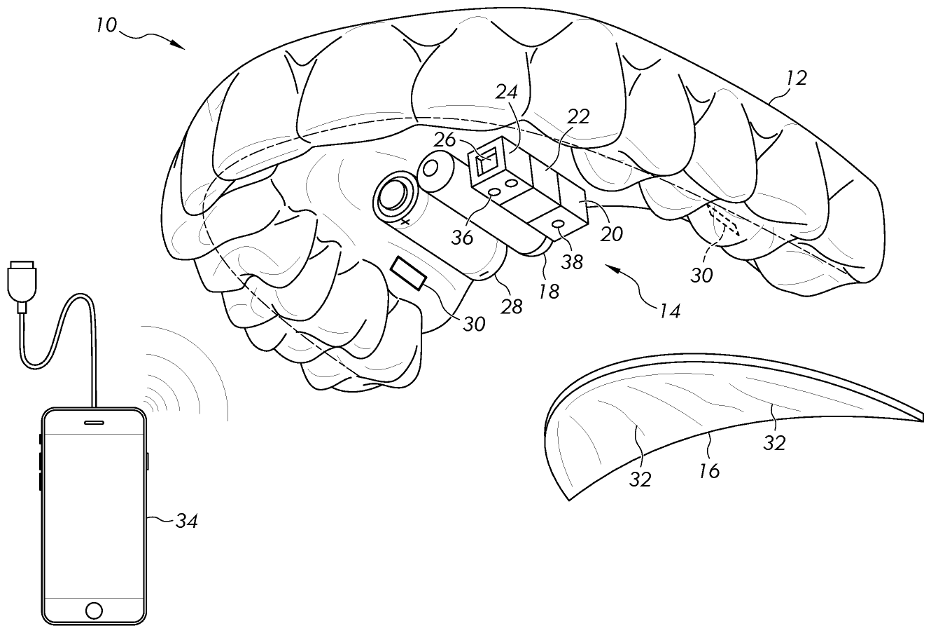

### FIELD OF INVENTION

The present application relates generally to an oral device. More particularly, it relates to an oral device configured stimulate a user to reduce or mitigate snoring and/or sleep apnea, for example.

### BACKGROUND OF INVENTION

In some cases, snoring and/or sleep apnea is caused by muscles and tissues that are unsupported by bone structure in the back of a person's mouth and throat, which relax into the upper airway and result in a decreased airway. This decreased airway increases the air velocity flowing through the airway while a person is breathing and causes the relaxed muscles and tissues, such as the tongue, upper palate, etc., to vibrate. It is these vibrations that result in snoring and/or sleep apnea. Various techniques, such as implantable stimulation devices and external devices (i.e., CPAP machines), are known to reduce or mitigate snoring and/or sleep apnea. Other methods include intraoral devices that shift a user's lower jaw forward or displace a user's tongue to maintain a position away from the back of the throat to widen the airway.

These known methods can cause various issues for a user. Some of these issues include user discomfort and pain, nocturnal salivation, dislodgement of the device, choking on the device, teeth displacement/deformation due to tension forces imposed on a user's teeth by the device, and/or user refusal due to the bulky and uncomfortable nature of the device. Thus, it is desirable to provide a discreet device that effectively displaces relaxed muscles and tissues out of the user's airway.

### BRIEF SUMMARY OF THE INVENTION

The following presents a simplified summary of the invention in order to provide a basic understanding of some aspects of the invention. The summary does not represent an extensive overview of invention, nor is the summary intended to identify key or critical elements of the invention or delineate the scope of the invention. The sole purpose of the summary is to present certain concepts of the invention in a simplified form as a prelude to the description of the invention that is presented hereinafter.

In accordance with a first aspect, an oral device includes a mouthpiece and a stimulant package comprising a motor, wherein the motor is configured to cause the mouthpiece to vibrate.

In accordance with a second aspect, a method of inhibiting snoring and/or sleep apnea includes placing an oral device comprising a mouthpiece and a stimulant package comprising a vibrating motor in a user's mouth prior to sleeping and activating the motor to cause the mouthpiece to vibrate within the user's mouth when a specified condition is met.

In accordance with a third aspect, a method of waking a user includes setting a parameter including a time of day; placing an oral device comprising a mouthpiece and a stimulant package comprising a vibrating motor in a user's mouth prior to sleeping; and activating the motor to cause the mouthpiece to vibrate within the user's mouth at the time of day.

### BRIEF DESCRIPTION OF THE DRAWINGS

The foregoing and other aspects of the present disclosure will become apparent to those skilled in the art to which the present disclosure relates upon reading the following description with reference to the accompanying drawings, in which:

The FIGURE is a schematic perspective view of an example oral device.

### DETAILED DESCRIPTION

Example embodiments that incorporate one or more aspects of the apparatus and methodology are described and illustrated in the drawings. These illustrated examples are not intended to be a limitation on the present disclosure. For example, one or more aspects of the disclosed embodiments can be utilized in other embodiments and even other types of devices. Moreover, certain terminology is used herein for convenience only and is not to be taken as a limitation.

Herein, when a range such as 5-25 (or 5 to 25) is given, this means preferably at least 5 and, separately and independently, preferably not more than 25. In an example, such a range defines independently at least 5, and separately and independently, not more than 25.

The present invention will now be described with reference to the drawing. Drawings are not necessarily drawn to scale within a given FIGURE. Also, the sizes of the components may be schematically drawn in order to facilitate an understanding of the drawings. In the following description, numerous specific details are set forth in order to provide a thorough understanding of the present invention, but it can be possible in certain instances to practice the present invention without those specific details. Additionally, the examples discussed are not intended to be a limit on the invention. For example, one or more aspects of the disclosed examples can be utilized in other examples and even other types of apparatus and devices.

Referring to the FIGURE, there is illustrated an example embodiment of the present invention comprising an oral device configured to be installed in the mouth of a user as an intraoral device. In one embodiment, the oral device comprises a mouthpiece 10. The mouthpiece can be shaped to receive a dentition of user, for example, the upper dentition. In one embodiment, the mouthpiece is custom-fit for a user by a dentist, orthodontist, or other suitable licensed professional. In another embodiment, the mouthpiece can be fit by a user with an in-home kit. The in-home kit can include a mouthpiece made of a formable or thermoformable material. In another embodiment, the mouthpiece can include a one-size-fits-all configuration, or a pre-sized (e.g., small, medium large, etc.) configuration.

The mouthpiece 12 material can include materials such as acrylic resins, polyurethane, thermoplastics, silicone, or any suitable soft rubber or rubber-like material, or a combination thereof. In some embodiments, the mouthpiece includes a pattern of different materials. The material of the mouthpiece can be clear, transparent, colored, and/or opaque plastic. In another embodiment, the mouthpiece can further include metal wire, including copper, nickel, titanium, or a combination thereof. In some embodiments, the plastic mouthpiece is fit to be received by the teeth of a user. In other embodiments, the metal wire and plastic mouthpiece is fit to be received by the teeth of a user.

In one embodiment, the mouthpiece measures between 2 cm and 12 cm, preferably between 4 cm and 10 cm, more preferably between 5 cm and 9 cm, and most preferably between 6 cm and 8 cm from the front to the back of the mouthpiece. The largest width from one side to the other side of the mouthpiece measures between 1 cm and 10 cm, preferably between 2 cm and 9 cm, more preferably between 3 cm and 7 cm, and most preferably between 4 cm and 6 cm. In some embodiments, the thickness of the mouthpiece measures between 0.5 mm and 10 mm, preferably between 0.75 mm and 9 mm, more preferably between 1 mm and 8 mm, and most preferably between 1.5 mm and 7 mm at its thinnest portion.

The oral device 10 further includes a stimulant package 14. In one embodiment, the stimulant package is permanently affixed to the mouthpiece wherein the mouthpiece 12 and stimulant package 14 are sealed such that the seal is waterproof. In another embodiment, the stimulant package is removably coupled to the mouthpiece with a cover 16, such that a user can remove and replace the stimulant package. In this embodiment, the cover 16 is sealed on the stimulant package 14 with a waterproof seal. The stimulant package can be custom-fit, one-size-fits-all, or pre-sized (e.g., small, medium, or large).

In one embodiment, the stimulant package 14 includes a stimulant 18 configured to incite movement of muscles and/or tissues within a user's mouth. In one embodiment, the stimulant package further includes at least one sensor 20 selected from the group comprising a contact sensor, pressure sensor, a sound sensor, an airflow sensor, an oxygen sensor, a vibration sensor, or a combination thereof. In another embodiment, the stimulant package includes a controller 22. The controller can include a transceiver 24 configured to wirelessly communicate with a remote device (i.e., a mobile device) with Bluetooth Wi-Fi, and/or any other wireless method. The stimulant package 14 can include a connecter 26, such as an electrical socket. The connector includes a sealable port configured to be open and able to receive a wire (via a mating connector) and to be closed when a wire is not connected. The port can be closed with a pivoting or hinged flap configured to create a waterproof seal when closed. The transceiver 24 and/or connector 26 enable external connections to components of the stimulant package 14. A power source, such as a battery 28, is also found within the stimulant package to provide power to the components.

In one example, the battery 28 can be rechargeable either with or without a wire. The rechargeable battery can be wirelessly charged as an integral or separate part of the stimulant package. The wireless charger can be associated with a universal charging pad and/or with a charging pad unique to the device. Alternatively, the battery can be recharged by an AC or DC power source via a wire connected to the connector 26. In another alternative, a photocell may be attached to or integrated into the mouthpiece for charging the battery. In another example, the battery can be a single-use battery. The battery can include an alkaline battery, a button cell battery, a lithium-ion battery, nickel cadmium battery, nickel-metal hydride battery, or any combination thereof.

The stimulant 18 can include at least one motor, at least one electronic stimulus, surface variation, or a combination thereof. In one embodiment, the motor produces vibrations that irritate a user enough to move the obstructing intraoral muscle or tissue (i.e., a user's tongue) to open the airway, but not wake the user. An electronic stimulus or plurality of electronic stimuli can be disposed such that they come into contact with a user's tongue, palate, or other intraoral muscle or tissue causing obstruction of the airway. In one example, exposed electrodes on the tongue or palatine surface of the mouthpiece impose electricals stimulation on a tongue or palate. In another example, surface variations 32 can be positioned (on the cover 16, for example) to irritate the user's tongue upon contact with the tongue to cause the user to reflexively move the tongue out of the user's airway.

In one embodiment, the motor is configured to vibrate. For example, the at least one motor can include an eccentric rotating mass vibration motor, a linear resonant actuator, or a combination thereof. A coin vibration motor or a shaftless motor is an example of an eccentric rotating mass vibration motor with no external parts. An eccentric mass vibration motor includes an eccentric mass to create an unbalanced force resulting in vibrations. In one embodiment, the eccentric mass vibration motor can be sealed in plastic and waterproof. Rather than using an eccentric mass to create force, a linear resonant actuator uses a magnetic mass attached to a spring and driven by a voice coil. In one embodiment, a motor includes a low profile that preferably measures from 1 mm to 20 mm, more preferably from 5 mm to 15 mm, and most preferably from 7 mm to 12 mm. In one example, the motor includes a profile size of 5 mm to 20 mm and is powered by 1 to 6 volts and 0.05 to 0.5 amps.

During use, the stimulant 18 can be activated to provide stimulation when a specified condition is met or specified conditions are met. For example, vibration caused by the motor can begin as a result of detected contact with a user's tongue or other intraoral muscle or tissue obstructing a user's airway. In another example, the vibration can begin upon detection of snoring vibration or sound by the sensor 20 in the mouthpiece or a remote sensor (such as a mobile phone, tablet, or dedicated remote controller), for example by a microphone 38 connected to the sensor 20 or in the remote sensor. In other embodiments, the vibration can be pre-programmed to vibrate at preset times. In some embodiments, once activated the vibration will occur for a length of time between 0.25 seconds and 5 seconds, preferably between 0.5 seconds and 4 seconds, more preferably between 0.75 seconds and 3 seconds, and most preferably between 1 second and 2 seconds. The speed of the motor is preferably between 100 Hz to 20000 Hz.

The electronic stimuli can receive power via at least one wire or wirelessly. In one embodiment, the electronic stimulus comprises one or more electrodes 30. For example, the device can include one electrode, two electrodes, three electrodes, four electrodes, or any number of electrodes suitable to stimulate a user's tongue, palate, or other intraoral muscle or tissue causing obstruction in the airway. In one embodiment, the electrode or electrodes are positioned between the middle and posterior region of the stimulant package and user's mouth. In another embodiment, the electrode or electrodes are disposed on the bottom surface or adjacent to the bottom surface to contact and/or stimulate the user's tongue. In one example, when activated, the electrode sends an electrical nerve stimulation to a user's tongue that is strong enough to move the tongue out of the airway, but not strong enough to wake the user. In some embodiments, the electrical stimulation pulses at a rate between 1 Hz and 10 Hz, preferably between 2 Hz and 8 Hz, more preferably between 3 Hz and 7 Hz, and most preferably between 4 Hz and 6 Hz.

In some embodiments, the bottom surface of the stimulant package or the surface adjacent to a user's tongue includes a surface variation 32. The surface variation can comprise ridges, rectangular projections, cylindrical projections, conical projections, or any other suitably shaped projections. The length of the projections can range from 0.50 mm to 10.0 mm, preferably from 1.0 mm to 8.00 mm, more preferably from 2.0 mm to 6.00 mm, and most preferably from 3.0 mm to 5.0 mm. At the widest part, the diameter of the projection ranges from 0.05 mm to 1.50 mm, preferably from 0.10 mm to 1.25 mm, more preferably from 0.15 mm to 1.00 mm, and most preferably from 0.25 mm to 0.75 mm. In some embodiments, the surface variation can include a plurality of ridges, bumps, or any other non-smooth variation configured to contact and irritate a user's tongue. The surface variation can be disposed near the front, middle, and/or rear portion of the device. In other embodiments, the variation spans the length of the mouthpiece. In one example, the projections comprise nylon, polypropylene, acrylic resins, polyurethane, thermoplastics, silicone, a soft rubber or rubber-like material, or a combination thereof. The surface variation can be integral with the device, or removably coupled to the device.

In other embodiments, the stimulant package further includes at least one sensor 20. The stimulant package can also include a data recorder configured to record information from the sensor and be in communication with the remote device. The at least one sensor can include a contact sensor, a pressure sensor, a sound sensor, an airflow sensor, an oxygen sensor, a vibration sensor, or a combination thereof embodied as a microphone, piezoelectric element, accelerometer, or the like. The sensor is configured to detect a condition or conditions at the sensor and the controller is configured to activate the stimulant package when a specified condition or conditions are met. The sensor can be integral to the stimulant package, removably coupled to the device, or incorporated into a remote device, such as a wireless communication device. When incorporated into a remote device, the sensor is in communication with the stimulator, such that upon a particular detection and signal by the sensor, the stimulator will activate. For example, a sound sensor can be incorporated into a remote device positioned in close proximity to a sleeping user. When snoring is detected, the sound sensor sends a signal to the stimulant package to activate the stimulator. As another example, the sensor, such as the vibration sensor or piezoelectric element can detect vibrations that indicate snoring and then the sensor sends a signal to the stimulant package to activate the stimulator. In another embodiment, the contact sensor can detect contact, such as tongue contact with the device. The pressure sensor can detect a change in pressure within a user's mouth, either based on a preset value or tracked and recorded pressure data unique to the user. Similarly, the airflow sensor activates the stimulator upon a reading of diminished airflow, compared to a preset value or a tracked and recorded value unique to the user.

The user can select the activation motor speed and/or electrical strength of stimulation. The user can also select the duration of stimulation. Once the appropriate parameters are set, a user can go to sleep. While sleeping, one sensor or a combination of sensors will send a signal to activate vibration and/or electrical stimulation upon detection of particular conditions. For example, if the sound sensor detects snoring or sound resembling snoring, then the sensor will send a signal to activate stimulation. If the pressure sensor detects a pressure change, then the sensor will send a signal to activate stimulation. If the contact sensor detects tongue contact, then the sensor will send a signal to activate stimulation. Once activated, the motor vibrates and/or the electrical stimulus sends an electrical signal and causes the tongue or other obstructing muscle or tissue to move out of the user's airway. In one embodiment, once the pressure difference, muscle contact, and/or snoring sound is no longer detected, the stimulation stops. In other embodiments, the stimulation occurs for a preset amount of time (e.g., 0.5, 1, 1.5, 2, 2.5, 3, 3.5, 4, 4.5, or 5 seconds). This cycle of stimulation occurs until the device is removed from a user's mouth and/or turned off. In some embodiments, the device can be turned off via the remote device. In other embodiments, an on/off switch is incorporated in the device. In yet another embodiment, the device automatically turns off upon being removed from a user's mouth and automatically turns on when it is placed into a user's mouth. Once removed, a user can wash the device. In some embodiments, the device can be washed with tap water. In other embodiments, the mouthpiece and/or stimulant package can be washed in a dishwasher. To charge the device for future use, the user can place the device on a charging pad and/or remove the waterproof plug and charge the device via a power cable.

In another embodiment, the stimulation can be activated from a remote device 34. The remote device can be connected to the stimulant package wirelessly via the transceiver 24 or by wire via the connector 26. The remote device can include a mobile phone or tablet, for example, with a specialized application configured to be self-learning and/or adaptive. The remote device can include the remote sensor, which can detect the conditions for activating the stimulant. Programming and/or control of the stimulation package may be performed at the oral device 10 via a user interface (such as buttons 36 on the controller 22) and/or the remote device 34. The controller 22 and/or the remote device 34 may include a processor, such as a microcomputer, mini-computer, Raspberry PI, Arduino, or similar, and may include a combination of different types of such devices connected by wire and/or wirelessly. In some embodiments, some functions of the device can be set or regulated the remote device 34 (such as the phone) or an intermediate device (such as the microcomputer). In other embodiments, these functions could be set or regulated by activating the user interface (such as soft touch buttons 36) on the oral device itself, by pressing the buttons with the tongue with a certain pattern or by biting down, for example in a certain pattern, on a user interface embodied as sensors incorporated into a biting surface of the oral device. For example, the application can recognize particular snoring sound characteristics of a principal user and distinguish between the sounds of a snoring sleep partner or pet, or any other snoring-like sounds (such as thunder or low frequency sound of a furnace, etc.). In other embodiments, the application can change the intensity and/or duration of the stimulation in real-time based on the user's reaction. For example, if an initial stimulation fails to result in the user clearing their airway, then the application can recognize that failure and increase the intensity and/or duration of a second stimulation, and so on until the user clears their airway. For example, software controlling the device can include artificial intelligence (AI) to recognize snoring, distinguish other sounds, and/or regulate the intensity and frequency of stimulation. AI software may be an integral part of the mouthpiece and/or the controlling device (mini processor or a wireless communication device). AI software also may be part of a cloud-based service, where the remote device the controller or a combination of the remote device and controller rely on cloud AI to process sound and recognize snoring in order to command a mouthpiece to stimulate the tongue or other oral tissues to clear the airflow obstruction. The system may include other sensors, such as a finger pulse oximetry unit to detect user oxygen desaturation, and may use data from the other sensors with data from the sensor 20 to adjust the stimulation of the user's mouth tissue by the mouthpiece and/or for other purposes.

In a further embodiment, the device can be used as an alarm to wake a user. For example, at a preselected time, the stimulator will activate and wake the user. In one example, the vibration motor activates and vibrates at a speed greater than the speed used to mitigate or inhibit snoring and/or sleep apnea. In another embodiment, the electrical stimulus or stimuli activate at a strength greater than the strength used to mitigate snoring and/or sleep apnea by stimulating the palate, the tongue, or any other intraoral tissue. In one example, a user can set the date and/or time via the remote device. The user can also select the stimulant separately such that the user can select the vibration motor, the electrical stimulus, or a combination thereof. In addition, the user and/or the associated mobile application or program can select the strength and/or speed of the stimulant.

To use the device as an alarm, a user sets the appropriate parameters via a remote device. For example, the remote device can include an application associated with the device that communicates with the device. A user can optionally set an alarm time, type of stimulation, speed or strength of stimulation, and/or length of time of stimulation. For example, a user can select the vibrating motor to vibrate for 5 seconds to occur at 6:30 AM. The options for vibration speed and/or electrical strength and the length of stimulation time allow for the device to accommodate all types of sleepers, like light and heavy sleepers.

Other possible functions of the device can include playing sounds from the cloud services or wireless communication device via a mini processor or wireless communication device, by the method of bone conduction (through teeth, palate, or jaw, where the mouthpiece is mechanically connected to conduct sound through the teeth, palate, or j aw), such as soothing nature sounds to facilitate falling asleep, or music or any other auditory material for entertainment or for alarm function or any other purposes. Software controlling device function, sound recognition, and data collection can be part of the oral device 10, the remote device 34, or both and enables device to function autonomously or with the help of Internet based cloud services. The software package may include a mobile device app, web-based app or any other means of software to control device locally and remotely (via Internet) and process sound and data.

The invention has been described with reference to the example embodiments described above. Modifications and alterations will occur to others upon a reading and understanding of this specification. Examples of embodiments incorporating one or more aspects of the invention are intended to include all such modifications and alterations insofar as they come within the scope of the appended claims.

What is claimed is:

1. An oral device comprising:
a mouthpiece; and
a stimulant package comprising a motor, wherein the motor is configured to cause the mouthpiece to vibrate.

2. The oral device of claim 1, the stimulant package further comprising a controller connected to control the motor.

3. The oral device of claim 2, the stimulant package further comprising at least one sensor connected to the controller.

4. The oral device of claim 3, the at least one sensor is selected from the group comprising: a microphone, a contact sensor, a pressure sensor, a sound sensor, an airflow sensor, an oxygen sensor, a vibration sensor, or a combination thereof.

5. The oral device of claim 3, the stimulant package further comprising a rechargeable battery connected to power the controller, sensor, and motor.

6. The oral device of claim 2, the stimulant package further comprising a transceiver connected to the controller and configured to communicate with a remote device by a wired or wireless connection.

7. The oral device of claim 6, wherein the controller is configured to receive programming, control, and/or condition information from the remote device.

8. The oral device of claim 2, wherein the controller is configured to activate the motor when a specified condition is met.

9. The oral device of claim 2, the stimulant package further comprising electrodes connected to the controller and configured to provide an electrical stimulation when a specified condition is met.

10. The oral device of claim 1, wherein the stimulant package is removably coupled to the mouthpiece.

11. The oral device of claim 1, the stimulant package further comprising a waterproof seal.

12. The oral device of claim 11, wherein the waterproof seal comprises a cover.

13. The oral device of claim 12, further comprising a plurality of projections extending from a surface of the cover.

14. A method of inhibiting snoring and/or sleep apnea comprising:
placing an oral device comprising a mouthpiece and a stimulant package comprising a vibrating motor in a user's mouth prior to sleeping; and
activating the motor to cause the mouthpiece to vibrate within the user's mouth when a specified condition is met.

15. The method of claim 14, further comprising detecting the specified condition using a sensor.

16. The method of claim 15, wherein the specified condition is detected based on conditions selected from the group comprising: contact, pressure, sound, airflow, vibration, oxygen, or a combination thereof.

17. The method of claim 14, further comprising receiving information about the specified condition from a remote device.

18. The method of claim 14, further comprising receiving programming and/or control information from a remote device.

19. The method of claim 18, wherein the programming and/or control information includes parameters including type of stimulation, speed of stimulation, strength of stimulation, duration of stimulation, or a combination thereof.

20. A method of waking a user comprising:
setting a parameter including a time of day;
placing an oral device comprising a mouthpiece and a stimulant package comprising a vibrating motor in a user's mouth prior to sleeping; and
activating the motor to cause the mouthpiece to vibrate within the user's mouth at the time of day.
